(19) United States
(12) Patent Application Publication
(10) Pub. No.: US 2019/0274861 A1
(43) Pub. Date: Sep. 12, 2019

(54) Posture Enhancing Garment

(71) Applicant: Alexandra Gitomer, Pittsburgh, PA (US)

(72) Inventor: Alexandra Gitomer, Pittsburgh, PA (US)

(21) Appl. No.: 16/292,462

(22) Filed: Mar. 5, 2019

Related U.S. Application Data
(60) Provisional application No. 62/639,242, filed on Mar. 6, 2018.

(51) Int. Cl. A61F 5/02; A61F 5/30; A41D 1/02; A41D 1/04; A41D 23/00

(52) U.S. Cl. CPC A61F 5/024; A61F 5/30; A41D 23/00; A41D 1/04; A41D 1/02

(57) ABSTRACT

A posture enhancing garment including a back panel that covers the back of the wearer, at least one pocket having a first portion positioned on the back panel in an area corresponding to the thoracic portion of the spine and a second portion positioned on the back panel in an area corresponding to a lumbar portion of the spine, a first support pad having a length along a major axis that is greater than a length along a minor axis, and a second support pad having a length along a major axis that is greater than a length along a minor axis. The first support pad is received in the first pocket such that the major axis of the first support pad extends substantially parallel to the spine, and the second support pad is received in the second pocket such that the major axis of the second support pad extends substantially perpendicular to the spine.

### CROSS REFERENCE TO RELATED APPLICATIONS

This application claims priority to U.S. Provisional Application No. 62/639,242 filed Mar. 6, 2018, the disclosure of which is hereby incorporated by reference in its entirety.

### BACKGROUND OF THE INVENTION

### Field of the Invention

This invention relates to a posture enhancing garment and, more specifically, to a posture enhancing garment having pockets into which removable pads may be inserted to support the wearer's spine and back.

### Description of Related Art

Proper posture when sitting, especially for long periods of time when working at a desk or a computer, can help to avoid muscle and skeletal discomfort both while sitting and after. Various back braces, orthopedic supports, and protective garments are available for protecting the spine and providing support to the spine, for example, the braces, orthopedic supports, and protective garments disclosed in U.S. Pat. Nos. 5,855,561; 6,687,920; and 7,784,116 and United States Patent Application Publication Nos. 2008/0060112; 2012/0144550; 2012/0174298; 2016/0128861; and 2017/0216633. However, none of these braces, supports, or protective garments are designed to specifically provide support to enhance posture during sitting to relieve muscle aches and fatigue. The present invention provides support in a convenient garment where the posture enhancing elements can be removed when they are no longer needed so that the garment may be comfortably worn at all times.

### SUMMARY OF THE INVENTION

The present invention is directed to a posture enhancing garment worn over the torso. The garment comprises a back panel configured to cover a back of the torso of the wearer and to extend from a neck of the wearer to a lower back of the wearer, at least one pocket having a first portion positioned on the back panel in an area corresponding to a thoracic portion of the spine of the wearer and a second portion positioned on the back panel in an area corresponding to a lumbar portion of the spine of the wearer, a first support pad having a major axis and a minor axis, a length of the first support pad along the major axis of the first support pad being greater than a length of the first support pad along the minor axis of the first support pad, and a second support pad having a major axis and a minor axis, a length of the second support pad along the major axis of the second support pad being greater than a length of the second support pad along the minor axis of the second support pad. The first support pad is received in the first portion of the at least one pocket and is oriented such that the major axis of the first support pad extends substantially parallel to an axis of the back panel that extends from a top of the back panel corresponding to the neck of the wearer to a bottom of the back panel corresponding to the lower back of the wearer, and the second support pad is received in the second portion of the at least one pocket and is oriented such that the major axis of the second support pad extends substantially perpendicular to the axis of the back panel. The garment may have two pockets one for receiving the first support pad and one for receiving the second support pad.

An outer surface of the first support pad may be convex, where the outer surface has at least one of a curvature extending parallel to the minor axis of the first support pad and perpendicular to the major axis of the first support pad and a curvature extending parallel to the major axis of the first support pad and perpendicular to the minor axis of the first support pad. At least one of the curvature of the outer surface of the first support pad extending parallel to the minor axis of the first support pad and perpendicular to the major axis of the first support pad and the curvature extending parallel to the major axis of the first support pad and perpendicular to the minor axis of the first support pad may have a substantially constant radius of curvature or may have a changing radius of curvature where a central portion of the outer surface of the first support pad has a larger radius of curvature than an outer portion of the outer surface of the first support pad. A first outer surface of the first support pad which contacts the back of the wearer may have a curvature extending parallel to the minor axis of the first support pad and perpendicular to the major axis of the first support pad and a curvature extending parallel to the major axis of the first support pad and perpendicular to the minor axis of the first support pad that each have a changing radius of curvature such that a central portion of the outer surface of the first support pad has a larger radius of curvature than an outer portion of the outer surface of the first support pad and a second outer surface of the first support pad that faces away from the back of the wearer may have a curvature extending parallel to the minor axis of the first support pad and perpendicular to the major axis of the first support pad and a curvature extending parallel to the major axis of the first support pad and perpendicular to the minor axis of the first support pad that each have a substantially constant radius of curvature.

A cross-section of the first support pad taken along the minor axis of the first support pad may be substantially oval, and a cross-section of the first support pad taken along the major axis of the first support pad is substantially oval.

The first support pad may comprise two pieces with a first piece providing a first outer surface of the first support pad which contacts the back of the wearer and a second piece providing a second outer surface of the first support pad that faces away from the back of the wearer. The first outer surface of the first support pad may have a curvature extending parallel to the minor axis of the first support pad and perpendicular to the major axis of the first support pad and a curvature extending parallel to the major axis of the first support pad and perpendicular to the minor axis of the first support pad that each have a changing radius of curvature such that a central portion of the first outer surface of the first support pad has a larger radius of curvature than an outer portion of the first outer surface of the first support pad, and the second outer surface may have a curvature extending parallel to the minor axis of the first support pad and perpendicular to the major axis of the first support pad and a curvature extending parallel to the major axis of the first support pad and perpendicular to the minor axis of the first support pad that each have a substantially constant radius of curvature.

The length of the first support pad along the major axis of the first support pad may be 1.1-20 times the length of the first support pad along the minor axis of the first support pad.

A first outer surface of the second support pad may be convex with at least one of a curvature extending parallel to the major axis of the second support pad and perpendicular to the minor axis of the second support pad and a curvature extending parallel to the minor axis of the second support pad and perpendicular to the major axis of the second support pad, and a second outer surface of the second support pad may be concave with at least one of a curvature extending parallel to the major axis of the second support pad and perpendicular to the minor axis of the second support pad and a curvature extending parallel to the minor axis of the second support pad and perpendicular to the major axis of the second support pad. At least one of the curvature of the first outer surface of the second support pad extending parallel to the major axis of the second support pad and perpendicular to the minor axis of the second support pad and the curvature of the first outer surface of the second support pad extending parallel to the minor axis of the second support pad and perpendicular to the major axis of the second support pad may have a substantially constant radius or may have a changing radius of curvature where a central portion of the first outer surface of the second support pad is curved while outer portions of the first outer surface of the second support pad are flatter and slope downward from the curved central portion. At least one of the curvature of the second outer surface of the second support pad extending parallel to the major axis of the second support pad and perpendicular to the minor axis of the second support pad and the curvature of the second outer surface of the second support pad extending parallel to the minor axis of the second support pad and perpendicular to the major axis of the second support pad may have a substantially constant radius or may have a changing radius of curvature. The second outer surface of the second support pad which contacts the back of the wearer may have a substantially constant radius of curvature, and the first outer surface of the second support pad may have a central portion having a convex curved surface where outer portions of the first outer surface of the second support pad are flatter and slope downward from a curved central portion.

A cross-section of the second support pad taken along the major axis may have an arc shape.

Alternatively, a first outer surface of the second support pad may be convex with at least one of a curvature extending parallel to the major axis of the second support pad and perpendicular to the minor axis of the second support pad and a curvature extending parallel to the minor axis of the second support pad and perpendicular to the major axis of the second support pad and a second outer surface of the second support pad may be convex with at least one of a curvature extending parallel to the major axis of the second support pad and perpendicular to the minor axis of the second support pad and a curvature extending parallel to the minor axis of the second support pad and perpendicular to the major axis of the second support pad.

### BRIEF DESCRIPTION OF THE DRAWINGS

FIG. 1 shows a perspective view of a garment according to the present invention;

FIG. 2 shows a perspective view of a thoracic support pad according to the present invention;

FIG. 3 shows a top view of the thoracic support pad of FIG. 2;

FIG. 4 shows a side view of the thoracic support pad of FIG. 2;

FIG. 5 shows a cross-sectional side view of the thoracic support pad of FIG. 2 taken along line A-A in FIG. 2;

FIG. 6 shows an end view of a thoracic support pad of FIG. 2;

FIG. 7 shows a cross-sectional end view of a thoracic support pad of FIG. 2 taken along line B-B in FIG. 2;

FIG. 8 shows a perspective view of the top piece of the thoracic support pad of FIG. 2;

FIG. 9 shows a top view of the top piece of the thoracic support pad of FIG. 8;

FIG. 10 shows a side view of the top piece of the thoracic support pad of FIG. 8;

FIG. 11 shows a cross-sectional side view of the top piece of the thoracic support pad of FIG. 8 taken along line C-C in FIG. 8;

FIG. 12 shows an end view of the top piece of the thoracic support pad of FIG. 8;

FIG. 13 shows a cross-sectional end view of the top piece of the thoracic support pad of FIG. 8 taken along line D-D in FIG. 8;

FIG. 14 shows a perspective view of the bottom piece of the thoracic support pad of FIG. 2;

FIG. 15 shows a top view of the bottom piece of the thoracic support pad of FIG. 14;

FIG. 16 shows a side view of the bottom piece of the thoracic support pad of FIG. 14;

FIG. 17 shows a cross-sectional side view of the bottom piece of the thoracic support pad of FIG. 14 taken along line E-E;

FIG. 18 shows an end view of the bottom piece of the thoracic support pad of FIG. 14;

FIG. 19 shows a cross-sectional end view of the bottom piece of the thoracic support pad of FIG. 14 taken along line F-F;

FIG. 20 shows a perspective view of a lumbar support pad according to the present invention;

FIG. 21 shows a top view of the lumbar support pad of FIG. 20;

FIG. 22 shows a side view of the lumbar support pad of FIG. 20;

FIG. 23 shows a cross-sectional side view of the lumbar support pad of FIG. 20 taken along line G-G;

FIG. 24 shows an end view of a lumbar support pad of FIG. 20; and

FIG. 25 shows a cross-sectional end view of the lumbar support pad of FIG. 20 taken along line H-H.

### DESCRIPTION OF THE INVENTION

For purposes of the description hereinafter, the terms “end”, “upper”, “lower”, “right”, “left”, “vertical”, “horizontal”, “top”, “bottom”, “lateral”, “longitudinal”, “proximal”, “distal” and derivatives thereof shall relate to the invention as it is oriented in the drawing figures. However, it is to be understood that the invention may assume various alternative variations and step sequences, except where expressly specified to the contrary. It is also to be understood that the specific devices and processes illustrated in the attached drawings, and described in the following specification, are simply exemplary embodiments or aspects of the invention. Hence, specific dimensions and other physical characteristics related to the embodiments or aspects disclosed herein are not to be considered as limiting. As used herein, unless otherwise expressly specified, all numbers such as those expressing values, ranges, amounts or percentages may be read as if prefaced by the word “about”, even if the term does not expressly appear. Any numerical range recited herein is intended to include all sub-ranges subsumed therein. For example, a range of “1 to 10” is intended to include any and all sub-ranges between and including the recited minimum value of 1 and the recited maximum value of 10, that is, all subranges beginning with a minimum value equal to or greater than 1 and ending with a maximum value equal to or less than 10, and all subranges in between, e.g., 1 to 6.3, or 5.5 to 10, or 2.7 to 6.1. Plural encompasses singular and vice versa. When ranges are given, any endpoints of those ranges and/or numbers within those ranges can be combined with the scope of the present invention. “Including”, “such as”, “for example” and like terms means “including/such as/for example but not limited to”.

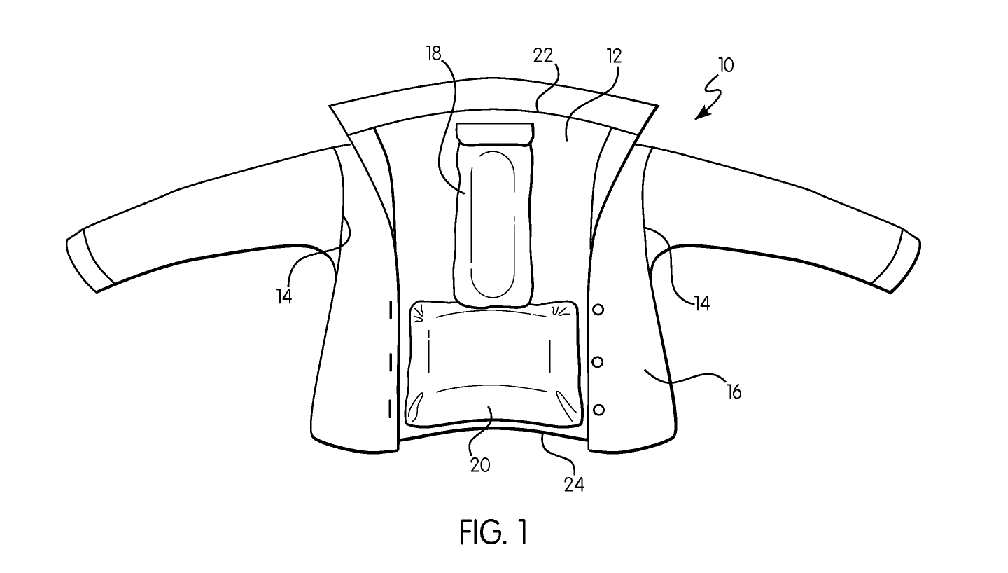

The present invention is directed to a posture enhancing garment 10 worn over the torso (FIG. 1). The garment 10 has a back panel 12 covering the back of the torso of the wearer and extending from the neck of the wearer to below the lower back of the wearer and a front portion 16 covering at least a portion of the front of the torso of the wearer. The back panel 12 and the front portion 16 may be a single piece or may be two or more pieces. For example, the back panel 12 may be a single piece and the front portion 16 may be two pieces as shown in FIG. 1. If the front portion 16 is two pieces, it may include at least one fastener for connecting the two pieces, for example, a zipper, buttons, Velcro®, snaps, hooks, or the like. The garment may also have openings 14 through which the wearer's arms may pass. As examples, the garment may be a jacket, a shirt, a pullover, a vest, a cape, a wrap, or a scarf.

The garment 10 has may have two pockets 18, 20 for receiving posture enhancing pads. The first pocket 18 is located at an upper portion of the back panel 12. The first pocket 18 is elongated such that it has a major axis and a minor axis, and the dimension (length) L₁ of the first pocket 18 along the major axis is larger than the dimension (width) W₁ of the first pocket 18 along the minor axis. The first pocket 18 extends from a first end 22 of the back panel 12 corresponding to the neck of the wearer and is centered along a central axis of the back panel 12. The major axis of the first pocket 18 is aligned parallel to and corresponds with the central axis of the back panel 12 such that the first pocket 18 is centered along the spine of the wearer. The length L₁ of the first pocket 18 extends along the central axis of the back panel 12 and along the thoracic region of the spine of the wearer, and the width W1 of the first pocket 18 extends transverse to the central axis of the back panel 12 and transverse to the spine of the wearer.

The second pocket 20 is located at a lower portion of the back panel 12. The second pocket 20 is elongated such that it has a major axis and a minor axis, and the dimension (length) L₂ of the second pocket 20 along the major axis is larger than the dimension (width) W₂ of the first pocket 18 along the minor axis. The second pocket 20 located adjacent a second end 24 of the back panel 12 corresponding to the lower back of the wearer. The major axis of the second pocket 20 is aligned transverse to the central axis of the back panel 12 such that the major axis of the second pocket 20 extends across the lower back of the wearer. The length L₂ of the second pocket 20 extends transverse to the central axis of the back panel 12 and across the lower back of the wearer, and the width W₂ of the second pocket 20 extends parallel to the central axis of the back panel 12 and parallel to the lumbar region of the spine of the wearer.

Alternatively, a single pocket having a T shape with a first portion having the features of the first pocket 18 and the second portion of the pocket having the features of the second pocket 20.

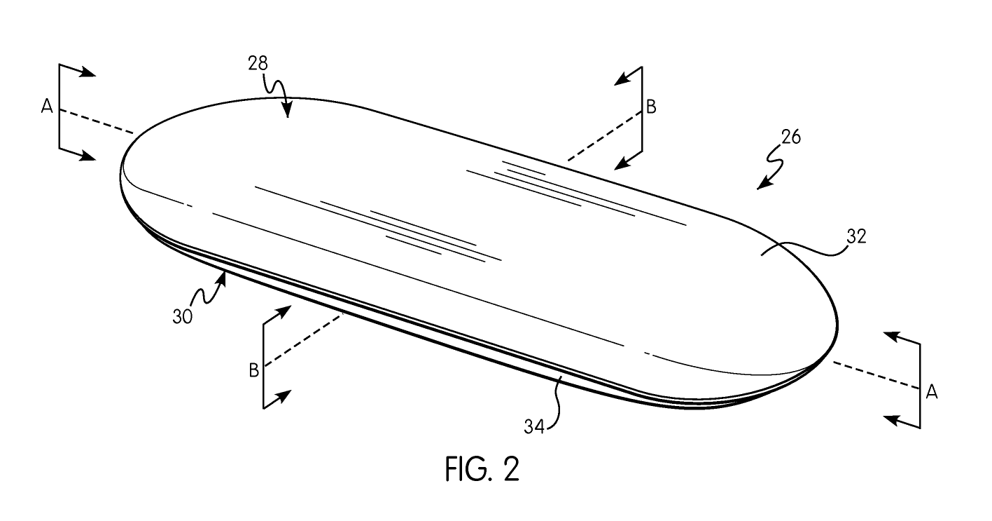

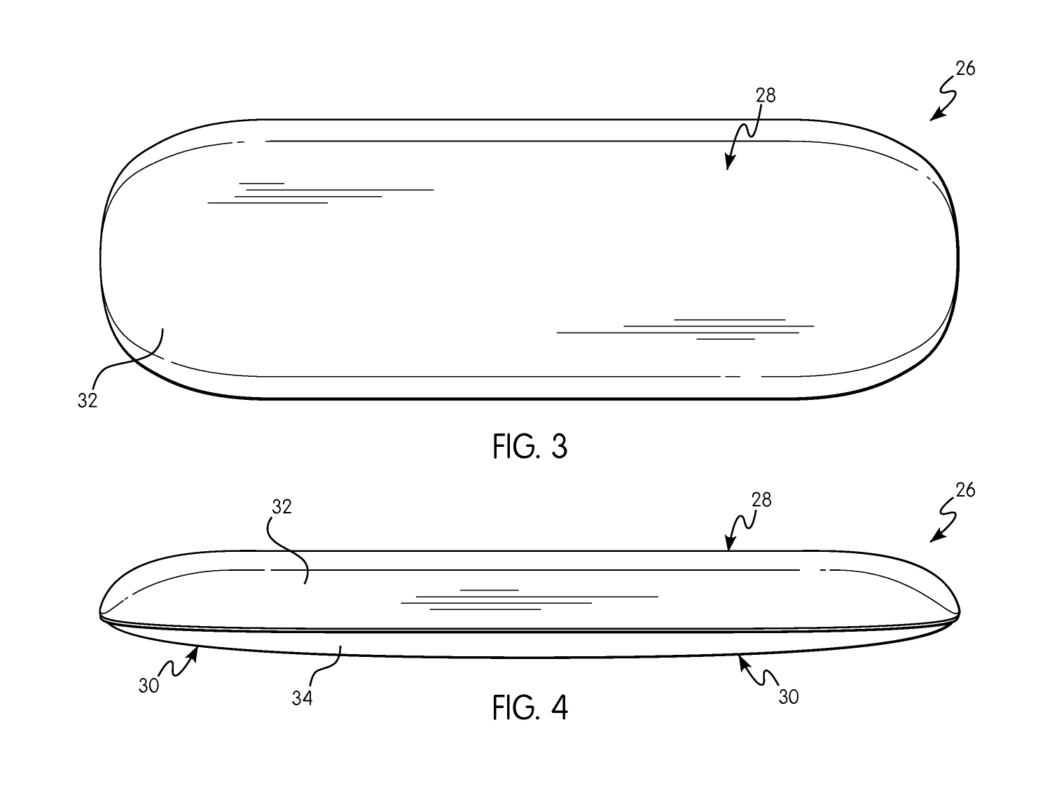

A thoracic support pad 26 (FIGS. 2-7) is provided for insertion into the first pocket 18. The thoracic support pad 26 is elongated such that it has a major axis and a minor axis, and the dimension (length) L₃ of the thoracic support pad 26 along the major axis is larger than the dimension (width) W₃ of the thoracic support pad 26 along the minor axis. For example, the thoracic support pad 26 may have the shape of a rectangle with rounded ends (FIG. 2), a rectangle, a rectangle with rounded corners, or an oval.

The thoracic support pad 26 is configured to lie along the thoracic region of the spine and between the shoulder blades (clavicles) of the wearer when the wearer's back contacts a hard surface, for example, the back of a chair. The shape of the thoracic support pad 26 supports the spine thereby providing room for the shoulder blades to rest further back allowing the wearer to rest comfortably with proper straight posture. The length L₃ of the thoracic support pad 26 is at least 1.1 times the width W₃ of the thoracic support pad 26 and up to 20 times the width W₃ of the thoracic support pad 26, for example, the length L₃ may be 1.1-20 times the width W₃, the length L₃ may be 1.5-15 times the width W₃, the length L₃ may be 2-10 times the width W₃, the length L₃ may be 2.5-5 times the width W₃, or the length L₃ may be 3 times the width W₃. For example, the length L₃ of the thoracic support pad 26 may be at least 2 inches and up to 30 inches, for example, 2-30 inches, 4-24 inches, or 6-18 inches, and the width W₃ of the thoracic support pad 26 may be at least 1 inch and up to 15 inches, for example, 1-15 inches, or 1.5-10 inches.

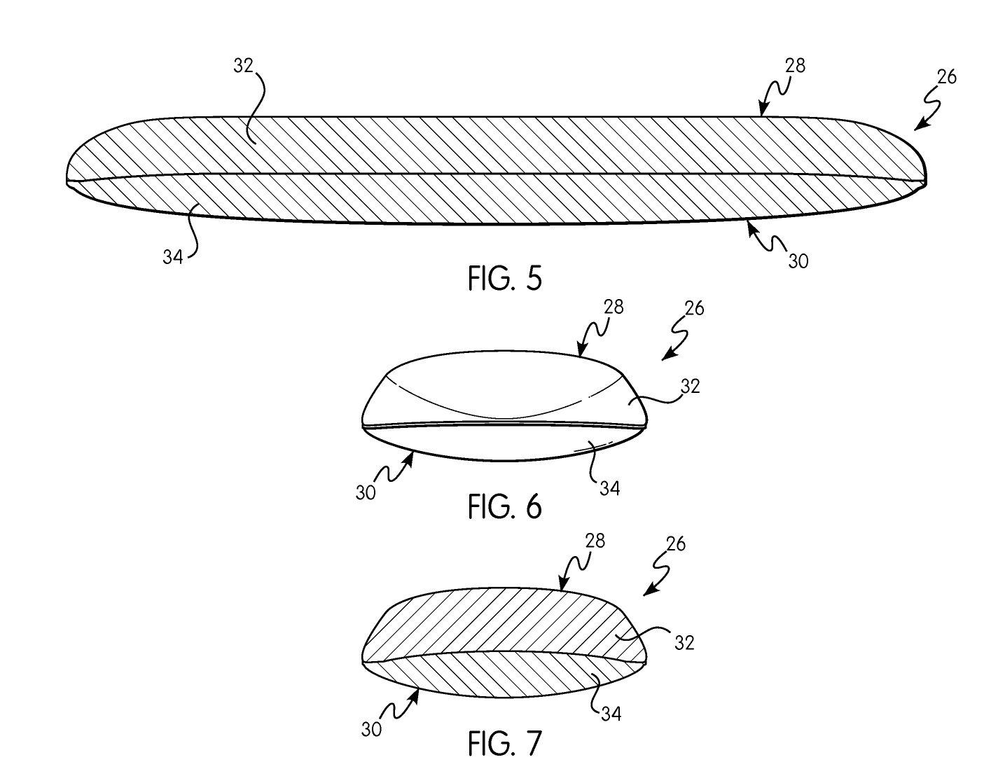

As shown in FIGS. 6 and 7, the outer surfaces 28, 30 of the thoracic support pad 26 are convex with a curvature extending parallel to the minor axis of the thoracic support pad 26 from one long side of the thoracic support pad 26 to the other long side of the thoracic support pad 26. For example, the cross-section of the thoracic support pad 26 taken along the minor axis may be substantially oval as shown in FIG. 7. The convex outer surfaces 28, 30 of the thoracic support pad 26 may have a substantially constant radius of curvature or may have a changing radius of curvature such that a central portion of the outer surfaces 28, 30 of the thoracic support pad 26 is less convex that outer portions of the outer surfaces 28, 30 of the thoracic support pad 26, i.e., the central portion of the outer surfaces 28, 30 has a larger radius of curvature than outer portions of the outer surfaces 28, 30 of the thoracic support pad 26. For example, the first outer surface 28 which contacts the wearer's back may have a changing radius of curvature extending parallel to the minor axis of the thoracic support pad 26 such that a central portion of the thoracic support pad 26 is less convex that outer portions of the outer surfaces 28, 30 of the thoracic support pad 26, i.e., the central portion of the outer surfaces 28, 30 has a larger radius of curvature than outer portions of the outer surfaces 28, 30 of the thoracic support pad 26, and the second outer surface 30 may have a substantially constant radius of curvature extending parallel to the minor axis of the thoracic support pad 26 as shown in FIGS. 4 and 5.

As shown in FIGS. 4 and 5, the outer surfaces 28, 30 of the thoracic support pad 26 may also be convex with a curvature extending parallel to the major axis of the thoracic support pad 26 from one short side of the thoracic support pad 26 to the other short side of the thoracic support pad 26. For example, the cross-section of the thoracic support pad 26 taken along the major axis may be substantially oval as shown in FIG. 5. The convex outer surfaces 28, 30 of the thoracic support pad 26 may have a substantially constant radius of curvature in this direction or may have a changing radius of curvature such that a central portion of the outer surfaces 28, 30 of the thoracic support pad 26 has a larger radius of curvature than outer portions of the outer surfaces 28, 30 of the thoracic support pad 26 in this direction. For example, the first outer surface 28 which contacts the wearer's back and the second outer surface 30 may have a changing radius of curvature extending parallel to the major axis of the thoracic support pad 26 such that a central portion of each of the outer surfaces 28, 30 of the thoracic support pad 26 is less convex than outer portions of the outer surfaces 28, 30 of the thoracic support pad 26, i.e., the central portion of the outer surfaces 28, 30 has a larger radius of curvature than outer portions of the outer surfaces 28, 30 of the thoracic support pad 26 as shown in FIGS. 6 and 7.

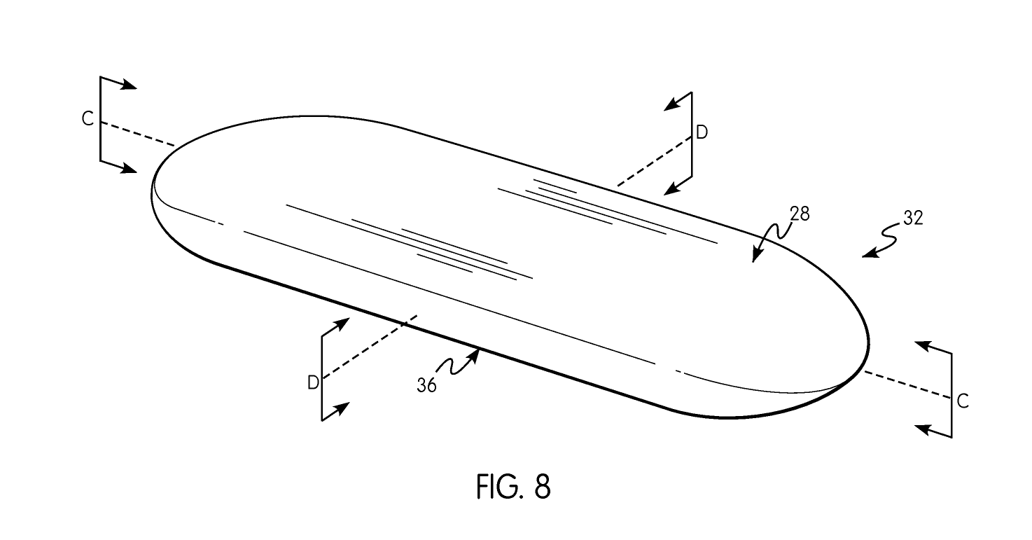

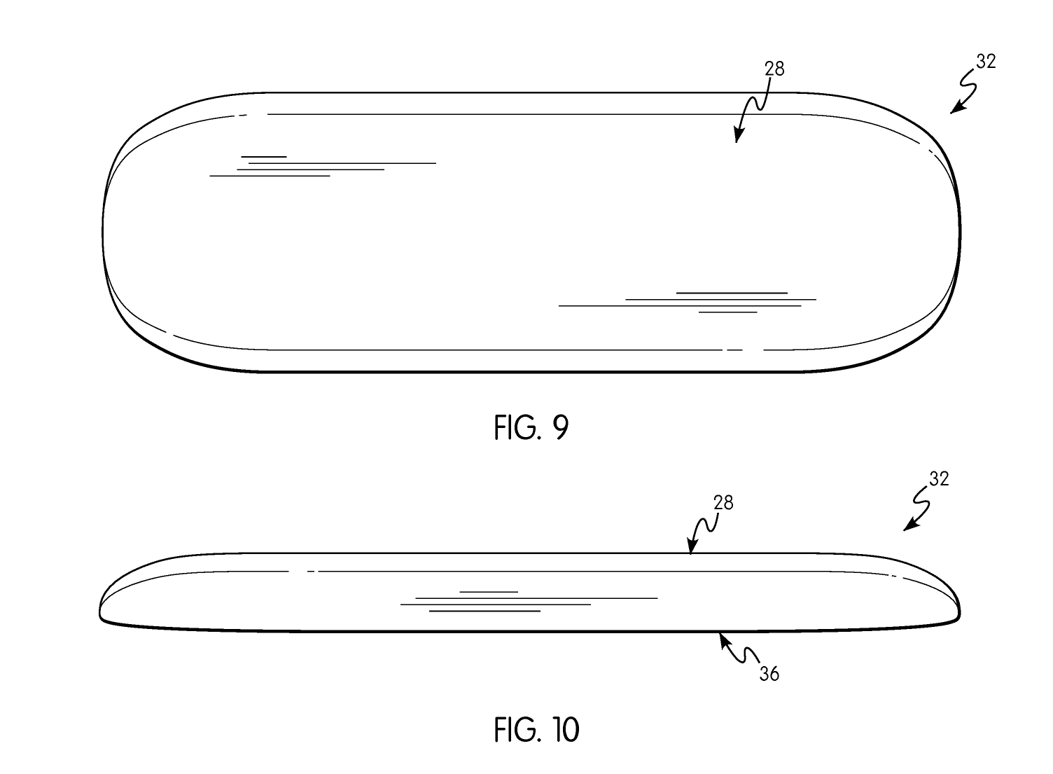

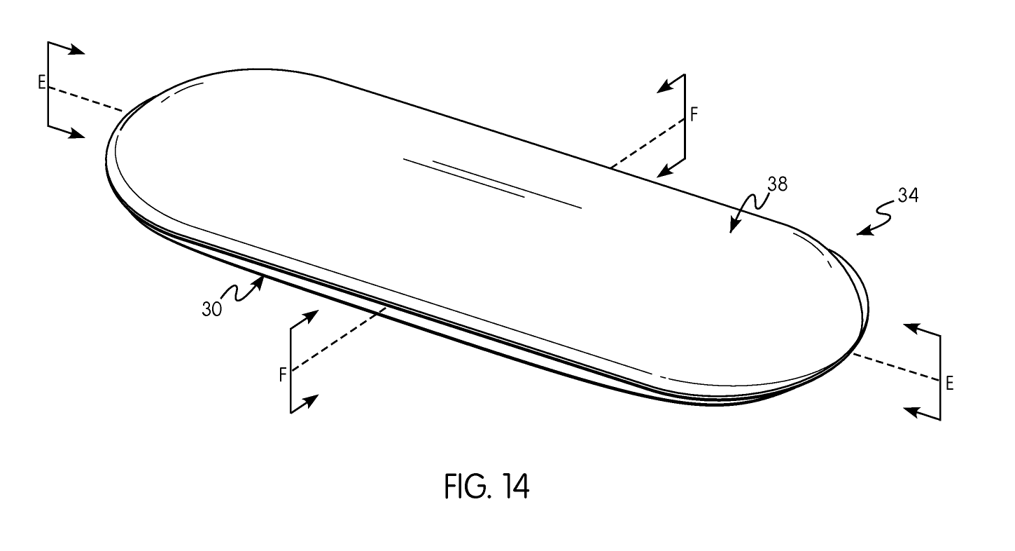

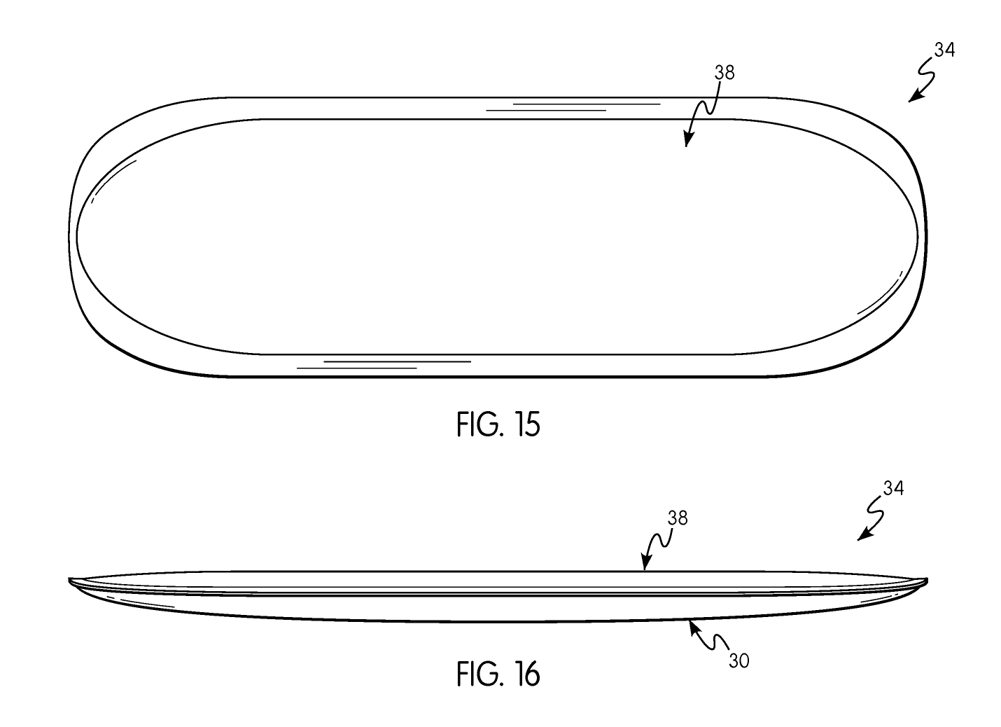

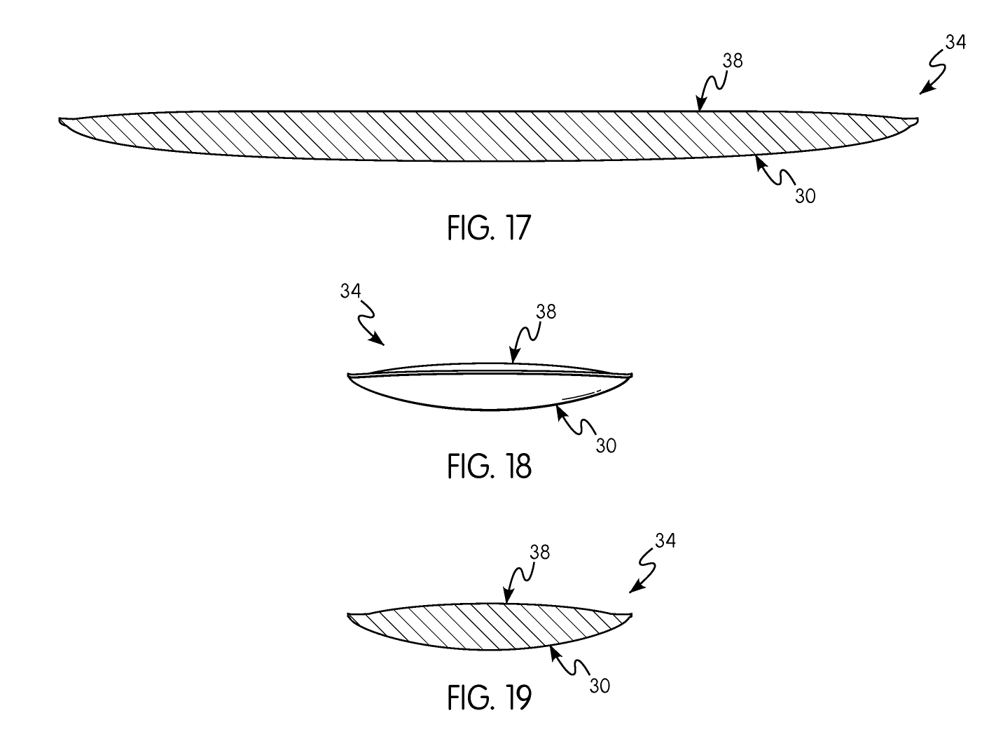

The thoracic support pad 26 may comprise a single piece or may comprise two pieces with the first piece 32 (FIGS. 8-13) providing the first outer surface 28 of the thoracic support pad 26 and the second piece 34 (FIGS. 14-19) providing the second outer surface 30 of the thoracic support pad 26. The outer surfaces of the first piece 32 and the second piece 34 combine to provide the thoracic support pad 26 with the curvatures described above.

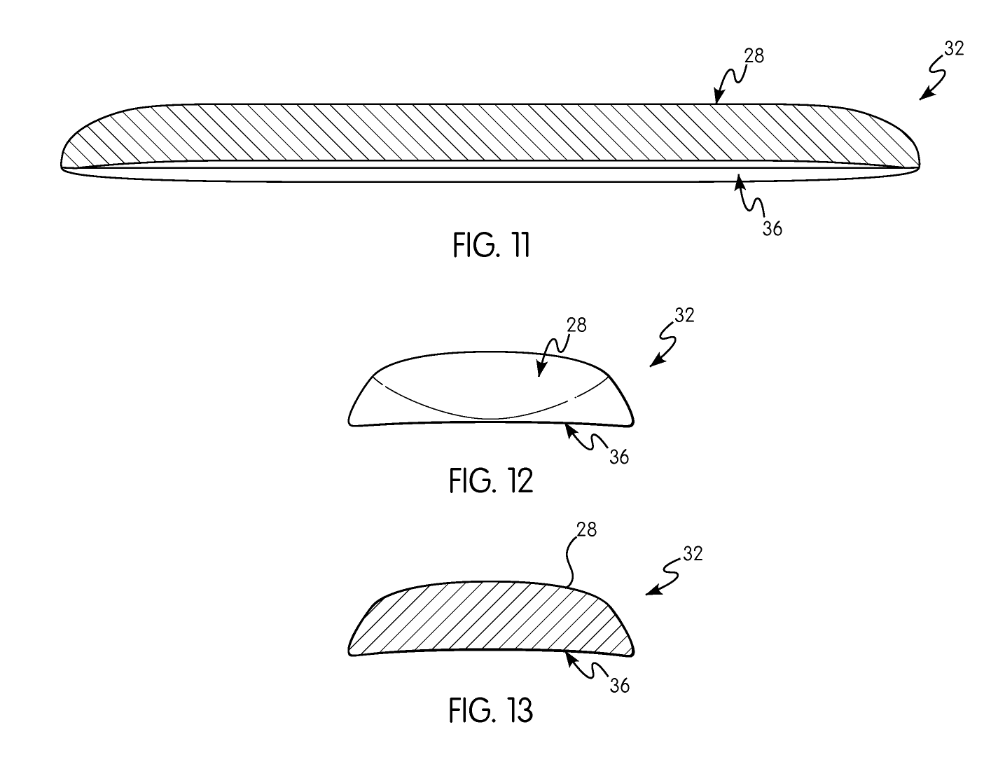

The first piece 32 has a first mating surface 36 that is opposite the first outer surface 28, and the second piece 34 has a second mating surface 38 that is opposite the second outer surface 30. The first mating surface 36 and the second mating surface 38 may be connected to one another using any suitable method, for example, gluing or thermal bonding. The first mating surface 36 and the second mating surface 38 may be substantially flat or one of the mating surfaces 36, 38 may be convex while the other mating surface 36, 38 is concave. For example, as shown in FIGS. 11-13 and 17-19, the first mating surface 36 may be concave to accept a second mating surface 38 that is convex.

The thoracic support pad 26 may be made of any suitable material, for example, polyurethane foam, including viscoelastic polyurethane foam and low-resilience polyurethane foam (LRPu) commonly referred to as memory foam, or polyethylene foam. For example, the first piece 32 of the thoracic support pad 26 that provides the first outer surface 28 of the thoracic support pad 26 which faces the back of the wearer may be made from memory foam while the second piece 34 of the thoracic support pad 26 that provides the second outer surface 30 of the thoracic support pad 26 which faces away from the back of the wearer may be made from polyethylene foam that is more stiff and less compressive than the memory foam from which the first piece of the thoracic support pad 26 is made.

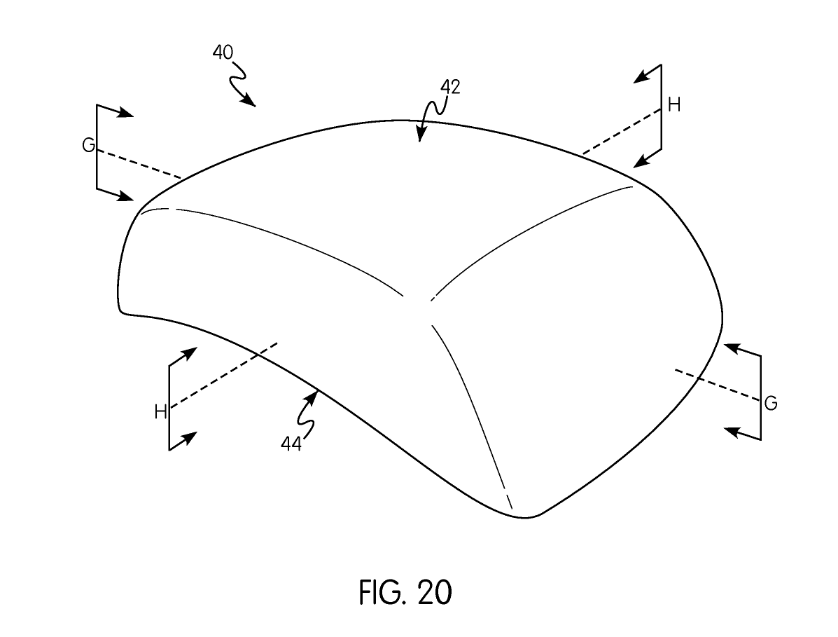

A lumbar support pad 40 (FIGS. 19-25) is provided for insertion into the second pocket 20. The lumbar support pad 40 is elongated such that it has a major axis and a minor axis, and the dimension (length) L₄ of the lumbar support pad 40 along the major axis is larger than the dimension (width) W₄ of the lumbar support pad 40 along the minor axis. For example, the lumbar support pad 40 may have the shape of a rectangle with rounded corners (FIG. 20), a rectangle, a rectangle with rounded ends, or an oval.

The lumbar support pad 40 is configured to lie in the area corresponding to the lumbar region of the spine and support the lower back of the wearer when the wearer's back contacts a hard surface, for example, the back of a chair. The major axis of the lumbar support pad 40 when placed in the second pocket 20 extends transverse to the wearer's spine. The length L₄ of the lumbar support pad 40 is at least 1.1 times the width W₄ of the lumbar support pad 40 and up to 20 times the width W₄ of the lumbar support pad 40, for example, the length L₄ may be 1.1-20 times the width W₄, the length L₄ may be 1.25-15 times the width W₄, the length L₄ may be 1.5-10 times the width W₄, the length L₄ may be 1.5-5 times the width W₄, or the length L₄ may be 1.6 times the width W₄. For example, the length L₄ of the lumbar support pad 40 may be at least 6 inches and up to 30 inches, for example, 6-30 inches, 8-24 inches, or 10-18 inches, and the width W₄ of the lumbar support pad 40 may be at least 4 inches and up to 24 inches, for example, 4-24 inches, 6-20 inches, or 8-18 inches.

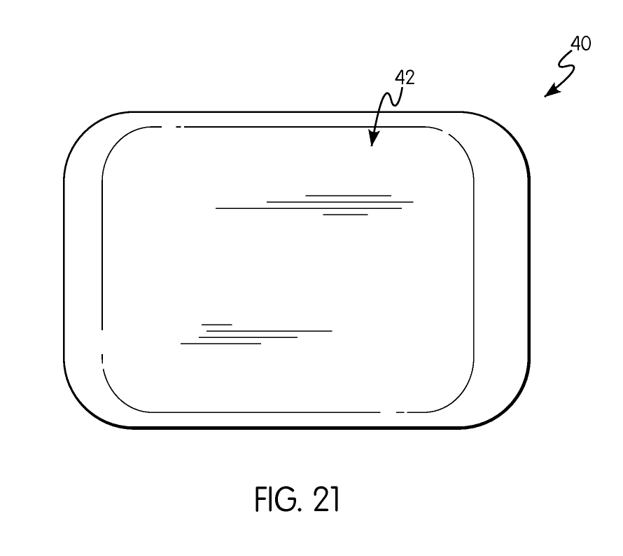

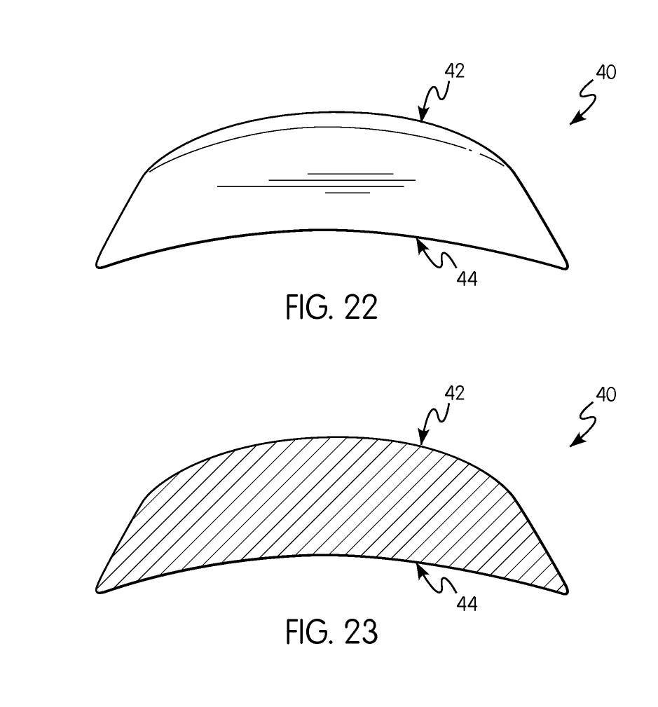

As shown in FIGS. 22 and 23, the first outer surface 42 of the lumbar support pad 40 is convex with a curvature extending parallel to the major axis of the lumbar support pad 40 from one short side of the lumbar support pad 40 to the other short side of the lumbar support pad 40, and the second outer surface 44 of the lumbar support pad 40 is concave with the curvature extending parallel to the major axis of the lumbar support pad 40 from one short side of the lumbar support pad 40 to the other short side of the lumbar support pad 40. For example, the cross-section of the lumbar support pad 40 taken along the major axis may have an arc shape as shown in FIG. 23. The convex first outer surface 42 of the lumbar support pad 40 may have a substantially constant radius of curvature or may have a changing radius of curvature. For example, the central portion of the first outer surface 42 of the lumbar support pad 40 may be curved while the outer portions of the first outer surface 42 of the lumbar support pad 40 are flatter and slope downward from the curved central portion. Likewise, the concave second outer surface 44 of the lumbar support pad 40 may have a substantially constant radius of curvature or may have a changing radius of curvature. For example, the second outer surface 44 of the lumbar support pad 40 which contacts the wearer's back may have a substantially constant radius of curvature extending parallel to the major axis, and the first outer surface 42 of the lumbar support pad 40 may have a central portion having a convex curve surface while the outer portions of the first outer surface 42 of the lumbar support pad 40 are flatter and slope downward from the curved central portion as shown in FIGS. 21 and 23.

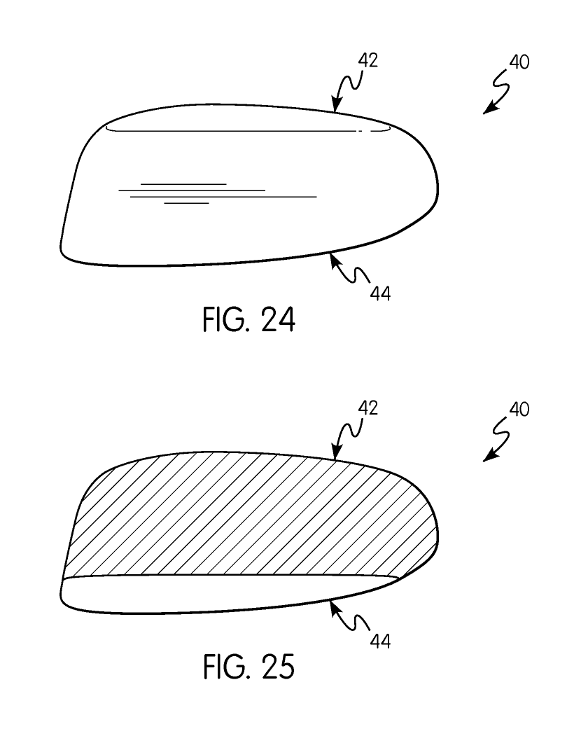

As shown in FIGS. 24 and 25, the first outer surface 42 of the lumbar support pad 40 may also be convex with a curvature extending parallel to the minor axis of the lumbar support pad 40 from one long side of the lumbar support pad 40 to the other long side of the lumbar support pad 40, and the second outer surface 44 of the lumbar support pad 40 may also be concave with a curvature extending parallel to the minor axis of the lumbar support pad 40 from one long side of the lumbar support pad 40 to the other long side of the lumbar support pad 40. For example, the cross-section of the lumbar support pad 40 taken along the minor axis may have the shape shown in FIG. 25. The convex first outer surface 42 of the lumbar support pad 40 may have a substantially constant radius of curvature in this additional direction or may have a changing radius of curvature in this additional direction. For example, the central portion of the lumbar support pad 40 may be curved while the outer portions of the lumbar support pad 40 are flatter and slope downward from the curved central portion. Likewise, the concave second outer surface 44 of the lumbar support pad 40 may have a substantially constant radius of curvature or may have a changing radius of curvature. For example, the second outer surface 44 of the lumbar support pad 40 which contacts the wearer's back may have a substantially constant radius of curvature extending parallel to the minor axis of the lumbar support pad 40, and the first outer surface 42 of the lumbar support pad 40 may have a central portion having a convex curved surface while the outer portions of first outer surface 42 of the lumbar support pad 40 are flatter and slope downward from the curved central portion at an almost 90° angle as shown in FIGS. 24 and 25.

Alternatively, similar to the thoracic support pad 26, the second outer surface 44 of the lumbar support pad 40 may be convex with the curvature extending parallel to the major axis of the lumbar support pad 40 from one short side of the lumbar support pad 40 to the other short side of the lumbar support pad 40. The convex second outer surface 44 of the lumbar support pad 40 may have a substantially constant radius of curvature or may have a changing radius of curvature where the outer portions of the second outer surface 44 of the lumbar support pad 40 are flatter and slope downward from the curved central portion. The second outer surface 44 of the lumbar support pad 40 may also be convex with a curvature extending parallel to the minor axis of the lumbar support pad 40 from one long side of the lumbar support pad 40 to the other long side of the lumbar support pad 40. The convex second outer surface 44 of the lumbar support pad 40, in this additional direction, may have a substantially constant radius of curvature or may have a changing radius of curvature, where outer portions of second outer surface 44 of the lumbar support pad 40 are flatter and slope downward from the curved central portion.

The lumbar support pad 40 may be made of any suitable material, for example, polyurethane foam including viscoelastic polyurethane foam and low-resilience polyurethane foam (LRPu) commonly referred to as memory foam or polyethylene foam. The lumbar support pad 40 may be made from the same material as the thoracic support pad 26 or may be made from a different material.

One or both of the thoracic support pad 26 and the lumbar support pad 40 may be enclosed in an air-tight membrane having a shape corresponding to the shape of the support pad 26, 40 and an opening through which air can pass. The membrane can be inflated or deflated by passing air through an opening in the membrane. The opening may include a valve that may be attached to a pump directly or via a hose. By introducing air into the membrane, the overall size of the support pads 26, 40 can be increased, and by removing air from the membrane, the overall size of the support pads 26, 40 can be decreased. If sufficient air is removed from the membrane, the support pad 26, 40 will be compressed and will stay compressed as long as the opening of the membrane is sealed. When the opening is unsealed, the support pad will automatically expand to its original size.

The thoracic support pad 26 may be removably placed in the first pocket 18 such that major axis of the thoracic support pad 26 extends parallel to the major axis of the first pocket 18 and the wearer's spine in the thoracic region of the spine and the minor axis of the thoracic support pad 26 extends parallel to the minor axis of the first pocket 18 and between the shoulder blades (clavicles) of the wearer. The lumbar support pad 40 may be removably placed in the second pocket 20 such that major axis of the lumbar support pad 40 extends parallel to the major axis of the second pocket 20 and transverse to the wearer's spine in the lumbar region of the spine, and the minor axis of the lumbar support pad 40 extends parallel to the minor axis of the second pocket 20 and the wearer's spine. Thus, the major axis of the thoracic support pad 26 is perpendicular to the major axis of the lumbar support pad 40. The first pocket 18 and the second pocket 20 may be placed such that the lower portion of the first pocket 18 contacts the upper portion of the second pocket 20 and the lower end of the thoracic support pad 26 contacts the upper end of the lumbar support pad 40 when the pads are placed in the pockets 18, 20.

The removable pads allow the garment to be comfortably worn when no posture enhancement is needed and to be laundered without any damage to the support pads. Further, the placement and shape of the support pads allows for optimum comfort and support of the wearer's spine and back when seated with their back resting against a hard object, such as a chair.

It is to be understood that the invention may assume various alternative variations, except where expressly specified to the contrary. It is also to be understood that the specific embodiments illustrated in the attached drawings, and described in the specification, are simply exemplary examples or aspects of the invention. Although the invention has been described in detail for the purpose of illustration based on what is currently considered to be the most practical and preferred embodiments or aspects, it is to be understood that such detail is solely for that purpose and that the invention is not limited to the disclosed embodiments or aspects, but, on the contrary, is intended to cover modifications and equivalent arrangements that are within the spirit and scope thereof. For example, it is to be understood that the present invention contemplates that, to the extent possible, one or more features of any embodiment or aspect can be combined with one or more features of any other embodiment or aspect.

1. A posture enhancing garment worn over the torso, the garment comprising:
a back panel configured to cover a back of the torso of the wearer and to extend from a neck of the wearer to a lower back of the wearer;
at least one pocket having a first portion positioned on the back panel in an area corresponding to a thoracic portion of the spine of the wearer and a second portion positioned on the back panel in an area corresponding to a lumbar portion of the spine of the wearer;
a first support pad having a major axis and a minor axis, a length of the first support pad along the major axis of the first support pad being greater than a length of the first support pad along the minor axis of the first support pad; and
a second support pad having a major axis and a minor axis, a length of the second support pad along the major axis of the second support pad being greater than a length of the second support pad along the minor axis of the second support pad;
wherein the first support pad is received in the first portion of the at least one pocket and is oriented such that the major axis of the first support pad extends substantially parallel to an axis of the back panel that extends from a top of the back panel corresponding to the neck of the wearer to a bottom of the back panel corresponding to the lower back of the wearer, and
the second support pad is received in the second portion of the at least one pocket and is oriented such that the major axis of the second support pad extends substantially perpendicular to the axis of the back panel.

2. The garment of claim 1, wherein the garment has two pockets one for receiving the first support pad and one for receiving the second support pad

3. The garment of claim 1, wherein an outer surface of the first support pad is convex, the outer surface having at least one of a curvature extending parallel to the minor axis of the first support pad and perpendicular to the major axis of the first support pad and a curvature extending parallel to the major axis of the first support pad and perpendicular to the minor axis of the first support pad.

4. The garment of claim 3, wherein at least one of the curvature of the outer surface of the first support pad extending parallel to the minor axis of the first support pad and perpendicular to the major axis of the first support pad and the curvature extending parallel to the major axis of the first support pad and perpendicular to the minor axis of the first support pad has a substantially constant radius of curvature.

5. The garment of claim 3, wherein at least one of the curvature of the outer surface of the first support pad extending parallel to the minor axis of the first support pad and perpendicular to the major axis of the first support pad and the curvature extending parallel to the major axis of the first support pad and perpendicular to the minor axis of the first support pad has a changing radius of curvature where a central portion of the outer surface of the first support pad has a larger radius of curvature than an outer portion of the outer surface of the first support pad.

6. The garment of claim 3, wherein a first outer surface of the first support pad which contacts the back of the wearer has a curvature extending parallel to the minor axis of the first support pad and perpendicular to the major axis of the first support pad and a curvature extending parallel to the major axis of the first support pad and perpendicular to the minor axis of the first support pad that each have a changing radius of curvature such that a central portion of the outer surface of the first support pad has a larger radius of curvature than an outer portion of the outer surface of the first support pad and a second outer surface of the first support pad that faces away from the back of the wearer has a curvature extending parallel to the minor axis of the first support pad and perpendicular to the major axis of the first support pad and a curvature extending parallel to the major axis of the first support pad and perpendicular to the minor axis of the first support pad that each have a substantially constant radius of curvature.

7. The garment of claim 1, wherein a cross-section of the first support pad taken along the minor axis of the first support pad is substantially oval.

8. The garment of claim 1, wherein a cross-section of the first support pad taken along the major axis of the first support pad is substantially oval.

9. The garment of claim 1, wherein the first support pad comprises two pieces with a first piece providing a first outer surface of the first support pad which contacts the back of the wearer and a second piece providing a second outer surface of the first support pad that faces away from the back of the wearer.

10. The garment of claim 9, wherein the first outer surface of the first support pad has a curvature extending parallel to the minor axis of the first support pad and perpendicular to the major axis of the first support pad and a curvature extending parallel to the major axis of the first support pad and perpendicular to the minor axis of the first support pad that each have a changing radius of curvature such that a central portion of the first outer surface of the first support pad has a larger radius of curvature than an outer portion of the first outer surface of the first support pad and the second outer surface has a curvature extending parallel to the minor axis of the first support pad and perpendicular to the major axis of the first support pad and a curvature extending parallel to the major axis of the first support pad and perpendicular to the minor axis of the first support pad that each have a substantially constant radius of curvature.

11. The garment of claim 1, wherein the length of the first support pad along the major axis of the first support pad is 1.1-20 times the length of the first support pad along the minor axis of the first support pad.

12. The garment of claim 1, wherein a first outer surface of the second support pad is convex with at least one of a curvature extending parallel to the major axis of the second support pad and perpendicular to the minor axis of the second support pad and a curvature extending parallel to the minor axis of the second support pad and perpendicular to the major axis of the second support pad and a second outer surface of the second support pad is concave with at least one of a curvature extending parallel to the major axis of the second support pad and perpendicular to the minor axis of the second support pad and a curvature extending parallel to the minor axis of the second support pad and perpendicular to the major axis of the second support pad.

13. The garment of claim 12, wherein at least one of the curvature of the first outer surface of the second support pad extending parallel to the major axis of the second support pad and perpendicular to the minor axis of the second support pad and the curvature of the first outer surface of the second support pad extending parallel to the minor axis of the second support pad and perpendicular to the major axis of the second support pad has a substantially constant radius.

14. The garment of claim 12, wherein at least one of the curvature of the first outer surface of the second support pad extending parallel to the major axis of the second support pad and perpendicular to the minor axis of the second support pad and the curvature of the first outer surface of the second support pad extending parallel to the minor axis of the second support pad and perpendicular to the major axis of the second support pad has a changing radius of curvature where a central portion of the first outer surface of the second support pad is curved while outer portions of the first outer surface of the second support pad are flatter and slope downward from the curved central portion.

15. The garment of claim 12, wherein at least one of the curvature of the second outer surface of the second support pad extending parallel to the major axis of the second support pad and perpendicular to the minor axis of the second support pad and the curvature of the second outer surface of the second support pad extending parallel to the minor axis of the second support pad and perpendicular to the major axis of the second support pad has a substantially constant radius.

16. The garment of claim 12, wherein at least one of the curvature of the second outer surface of the second support pad extending parallel to the major axis of the second support pad and perpendicular to the minor axis of the second support pad and the curvature of the second outer surface of the second support pad extending parallel to the minor axis of the second support pad and perpendicular to the major axis of the second support pad has a changing radius of curvature.

17. The garment of claim 12, wherein the second outer surface of the second support pad which contacts the back of the wearer has a substantially constant radius of curvature, and the first outer surface of the second support pad has a central portion having a convex curved surface where outer portions of the first outer surface of the second support pad are flatter and slope downward from a curved central portion.

18. The garment of claim 1, wherein a cross-section of the second support pad taken along the major axis has an arc shape.

19. The garment of claim 1, wherein a first outer surface of the second support pad is convex with at least one of a curvature extending parallel to the major axis of the second support pad and perpendicular to the minor axis of the second support pad and a curvature extending parallel to the minor axis of the second support pad and perpendicular to the major axis of the second support pad and a second outer surface of the second support pad is convex with at least one of a curvature extending parallel to the major axis of the second support pad and perpendicular to the minor axis of the second support pad and a curvature extending parallel to the minor axis of the second support pad and perpendicular to the major axis of the second support pad.
